(19) United States
(12) Patent Application Publication
(10) Pub. No.: US 2006/0269968 A1
(43) Pub. Date: Nov. 30, 2006

(54) Method of assessing the effectiveness of a treatment regimen

(75) Inventor: Alessio Fasano, West Friendship, MD (US)

Correspondence Address: Connolly Bove Lodge & Hutz LLP, Suite 800, 1990 M Street, N.W., Washington, DC, 20036, US

(73) Assignee: University of Maryland - Baltimore, Baltimore, MD (US)

(21) Appl. No.: 11/433,451

(22) Filed: May 15, 2006

Related U.S. Application Data
(60) Provisional application No. 60/680,868, filed on May 13, 2005.

(51) Int. Cl. G01N 33/567; G01N 33/53

(52) U.S. Cl. 435/7.2

(57) ABSTRACT

The present invention provides materials and methods to assess the efficacy of a treatment. In some embodiments, the present invention provides a method of assessing the efficacy of a treatment for celiac disease by measuring the serum zonulin level of a subject having received the treatment.

### CROSS REFERENCE TO RELATED APPLICATIONS

This application claims priority to U.S. provisional patent application Ser. No. 60/680,868 filed May 13, 2005, the contents of which are specifically incorporated herein by reference.

### FIELD OF THE INVENTION

The present invention relates to the field of diagnostics and evaluation of therapeutic modalities.

### BACKGROUND OF THE INVENTION

Mammalian epithelia contain structures referred to as zonula occludens (ZO) also referred to as tight junctions (TJs). These structures regulate the passage of materials through the epithelia by controlling access to the space between the epithelial cells (the paracellular pathway). To meet the many diverse physiological and pathological challenges to which epithelia are subjected, the tight junctions or zonula occludens must be capable of rapid, physiologic, reversible, transient, energy dependent, and coordinated responses that require the presence of a complex regulatory system. Examples of epithelia containing tight junctions include, but are not limited to, the intestines (particularly the small intestine), and the blood brain barrier.

In the absence of stimuli, the tight junctions are closed restricting access to the paracellular pathway. In the presence of stimuli, the tight junctions are reversibly opened. In U.S. Patent Nos. 5,945,510 and 5,948,629, novel mammalian proteins that function as the physiological modulator of mammalian tight junctions, have been identified and purified. These mammalian proteins, referred to as “zonulin,” function as the physiological effector of mammalian tight junctions. Certain bacteria have been shown to have toxins that stimulate the opening of tight junctions. Vibrio cholerae produces a toxin (zonula occludens toxin, ZOT) that has been shown to cause opening of tight junctions. It has been shown that 6 His-ΔG, an N-terminal deletion of ZOT in which the first 264 amino acids have been deleted and replaced with a six histidine purification tag, retains the ability to open tight junctions.

Intestinal tight junction dysfunction occurs in auto-immune diseases and in a variety of clinical conditions affecting the gastrointestinal tract, including food allergies, enteric infections, malabsorption syndromes such as celiac disease, and inflammatory bowel diseases. Healthy, mature gut mucosa with its intact tight junction serves as the main barrier to the passage of macromolecules. During the healthy state, small quantities of immunologically active proteins cross the gut host barrier. When the integrity of the tight junction system is compromised, as with prematurity or after exposure to radiation, chemotherapy, and/or toxins, a deleterious response to environmental antigens (including autoimmune diseases and food allergies) can occur.

Celiac disease (CD) is a condition in which ingestion of gluten (a protein found in wheat, rye, and barley) results in an abnormal increase in intestinal tight junction permeability. It is well known that celiac disease can be associated with other autoimmune diseases (AD). It is however still unclear whether the CD-associated risk of other AD is related to ongoing gluten ingestion or simply depends on common genetic background. Currently, the only treatment available for celiac disease is a gluten-free diet (GFD).

In order to evaluate the efficacy of a therapeutic agent in treating diseases associated with abnormal epithelial permeability, it would be useful to have a marker that changes with the progression of the disease. Zonulin, which is responsible for the modulation of intestinal permeability, is up regulated in CD and other AD, such as Type 1 diabetes. The present invention demonstrates that zonulin can serve as marker with which to evaluate the efficacy of a therapeutic agent in treating diseases associated with abnormal epithelial permeability.

### SUMMARY OF THE INVENTION

The present invention provides materials and methods for assessing the effectiveness of a treatment. Thus, in one embodiment, the present invention provides a method of assessing the effectiveness of a treatment of a condition in a subject in need of such treatment. Such methods typically comprise obtaining a sample from the subject and determining zonulin concentration in the sample. Samples may be of any type known to those skilled in the art. In one embodiment, a sample may be a blood sample. Samples may be processed by methods well known to those of skill in the art. In one embodiment, methods of the invention may comprise isolating serum from the sample and determining zonulin concentration in the serum.

The present invention may be used to evaluate the treatment of a wide variety of conditions. Typically, the present invention may be used to evaluate the treatment of a condition characterized by abnormal tight junction permeability. Thus, the present invention may be used to evaluate the effectiveness of a treatment for conditions associated with an increase in epithelial permeability. Treatments for conditions that may be evaluated include, but are not limited to, treatments for autoimmune diseases, treatments for celiac disease, and treatments for celiac associated diseases.

In one embodiment, methods of the invention may comprise determining zonulin concentration in a sample by methods involving the use of one or more antibodies, e.g., ELISAs. In one embodiment, methods of the invention may comprise contacting the sample with a first antibody that binds to zonulin under binding conditions, contacting the bound sample with a second antibody that binds zonulin under binding conditions; and detecting the presence of bound second antibody. In such methods, the first antibody may be fixed to a solid support, for example, a bead or a well in a microtiter plate. In some embodiments, at least one antibody may be raised against a protein comprising a fragment of zonula occludens toxin, for example, the first antibody may be raised against a protein comprising a fragment of zonula occludens toxin. A protein comprising any suitable fragment of zonula occludens toxin may be used, for example, a protein comprising ΔG fragment of zonula occludens toxin. In one embodiment, a protein comprising a suitable fragment of zonula occludens toxin may be a protein wherein the ΔG fragment comprises an N-terminal six histidine tag. In some methods of the invention, at least one antibody may be raised against a protein comprising zonula occludens toxin, for example, the second antibody may be raised against a protein comprising zonula occludens toxin. The second antibody may comprise one or more detectable moieties. In one embodiment, the second antibody may comprise biotin.

In one specific embodiment, the present invention provides a method for assessing the efficacy of a treatment for celiac disease in a subject in need of such treatment. Such a method may comprise obtaining a sample from the subject; and determining zonulin concentration in the sample. In some embodiments, the sample may be a blood sample. Such a method may further comprise isolating serum from the sample; and determining zonulin concentration in the serum. In some embodiments, determining zonulin concentration may comprise affixing a first antibody to a solid support, contacting the first antibody with sample under conditions causing zonulin in the sample to bind to the first antibody, contacting the bound zonulin with a second antibody under condition that cause the second antibody to bind zonulin bound to the first antibody, and detecting the bound second antibody. In some embodiments, the second antibody may comprise a detectable moiety. A detectable moiety may be directly detectable, for example, when the moiety is a fluorescent group. In some embodiments of the invention, a detectable moiety may be contacted with a reagent that binds to the detectable moiety. For example, a detectable moiety may be biotin and a reagent that binds to biotin (e.g., avidin or streptavidin conjugated to an enzyme such as alkaline phosphatase) may be added. When a reagent that binds to the detectable moiety comprises an enzyme, methods of the invention will typically include adding a substrate for the enzyme to the bound reagent and detecting conversion of the substrate into product either by detecting the product or by detecting the decrease in substrate.

The present invention also contemplates kits for evaluating the effectiveness of a treatment for a condition, for example, treatment of celiac disease. Such kits typically comprise means for detecting zonulin. Means for detecting zonulin may comprise one or more of anti-ΔG antibodies, biotinylated anti-ΔG antibodies, anti-ZOT antibodies, and biotinylated anti-ZOT antibodies, thus kits of the invention may comprise one or more containers containing one or more of anti-ΔG antibodies, biotinylated anti-ΔG antibodies, anti-ZOT antibodies, and biotinylated anti-ZOT antibodies. In some embodiments, a kit of the invention may comprise a container containing an antibody. Such antibodies may be anti-IgG antibodies, anti-IgA, anti-IgM antibodies, and/or anti-IgE antibodies. Antibodies for use in the kits of the invention may comprise one or more detectable moieties. In some embodiments, a kit of the invention may comprise a container containing zonulin and/or a container containing ΔG. Kits of the invention may comprise one or more protein binding partners of auto-antibodies. For example, kits of the invention may comprise one or more containers containing one or more of human transglutaminase (tTG), endomysial (EMA), tyrosine phosphates (IA-2), thyroid peroxidase (TPO), thyroglobulin (TG), islet cells or proteins therefrom, insulin, or glutamic acid decarboxylase (GAD).

The present invention also contemplates kits for evaluating the effectiveness of a treatment for celiac disease. Such kits typically comprise means for detecting zonulin. Means for detecting zonulin may comprise one or more of anti-ΔG antibodies, biotinylated anti-ΔG antibodies, anti-ZOT antibodies, and biotinylated anti-ZOT antibodies, thus kits of the invention may comprise one or more containers containing one or more of anti-ΔG antibodies, biotinylated anti-ΔG antibodies, anti-ZOT antibodies, and biotinylated anti-ZOT antibodies. In some embodiments, a kit of the invention may comprise a container containing an antibody. Such antibodies may be anti-IgG antibodies, anti-IgA, anti-IgM antibodies, and/or anti-IgE antibodies. Antibodies for use in the kits of the invention may comprise one or more detectable moieties. In some embodiments, a kit of the invention may comprise a container containing zonulin and/or a container containing ΔG. Kits of the invention may comprise one or more protein binding partners of auto-antibodies. For example, kits of the invention may comprise one or more containers containing one or more of human transglutaminase (tTG), endomysial (EMA), tyrosine phosphates (IA-2), thyroid peroxidase (TPO), thyroglobulin (TG), islet cells or proteins therefrom, insulin, or glutamic acid decarboxylase (GAD).

### BRIEF DESCRIPTIONS OF THE DRAWINGS

FIG. 1 is a bar graph showing the results of an assay for celiac disease biomarkers from celiac disease patients that complied with the GFD diet.

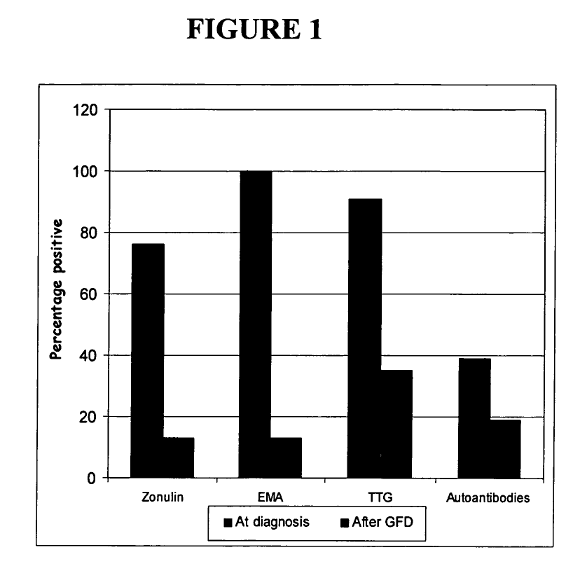

FIG. 2 is a bar graph showing the results of an assay for auto-antibodies from celiac disease patients that complied with the GFD diet.

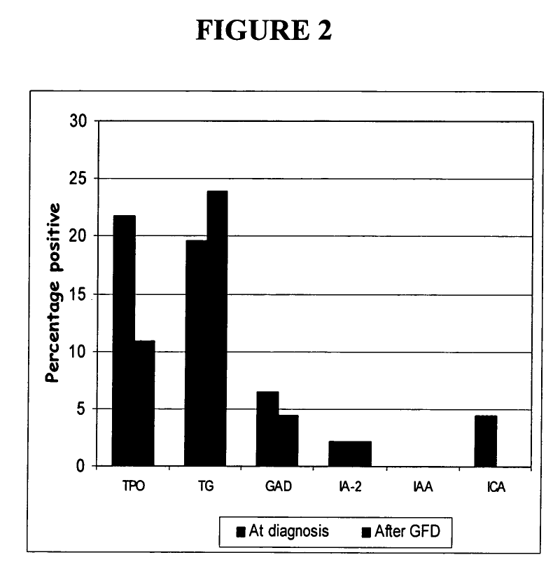

FIG. 3 is a bar graph showing the results of an assay for celiac disease biomarkers from celiac disease patients that did not comply with the GFD diet.

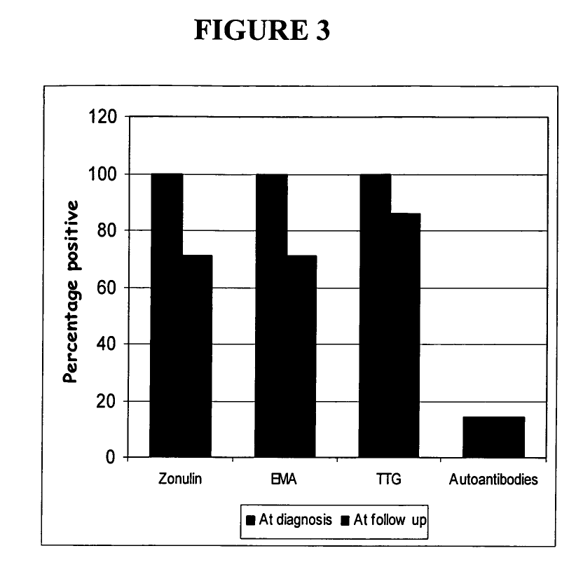

FIG. 4 is a bar graph showing the results of an assay for auto-antibodies from celiac disease patients that did not comply with the GFD diet.

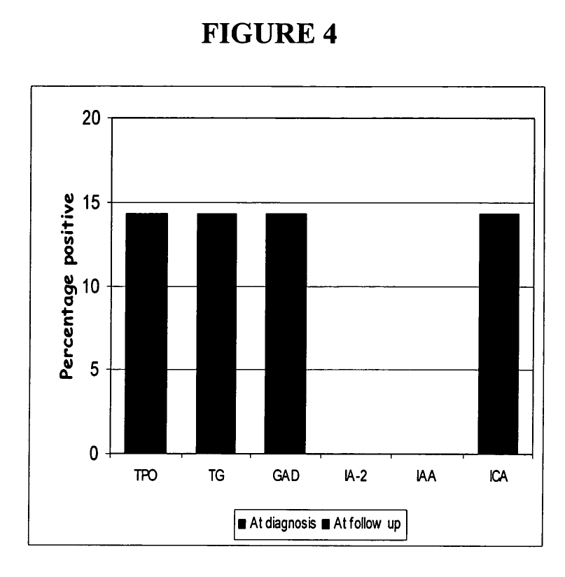

FIG. 5 is a bar graph showing the results of an assay for auto-antibodies from subjects having an associated Al condition showing the prevalence of auto-antibodies in relation to pre-existing autoimmune (AI) conditions (Type one diabetes, Graves' disease or Rheumatoid arthritis).

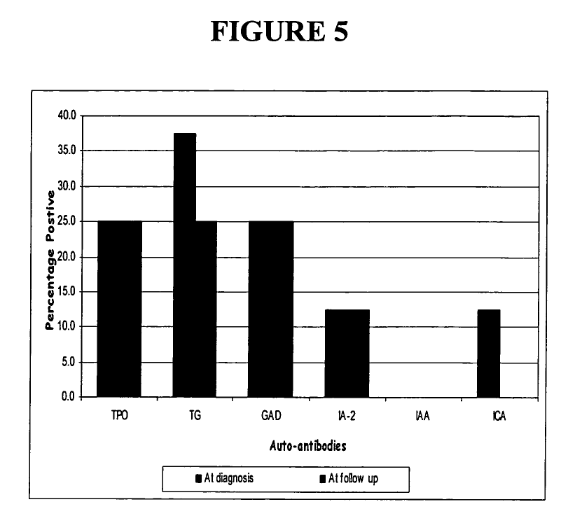

FIG. 6 is a bar graph showing the results of an assay for auto-antibodies from subjects not having an associated AI condition showing the prevalence of auto-antibodies in relation to pre-existing autoimmune (AI) conditions (Type one diabetes, Graves' disease or Rheumatoid arthritis).

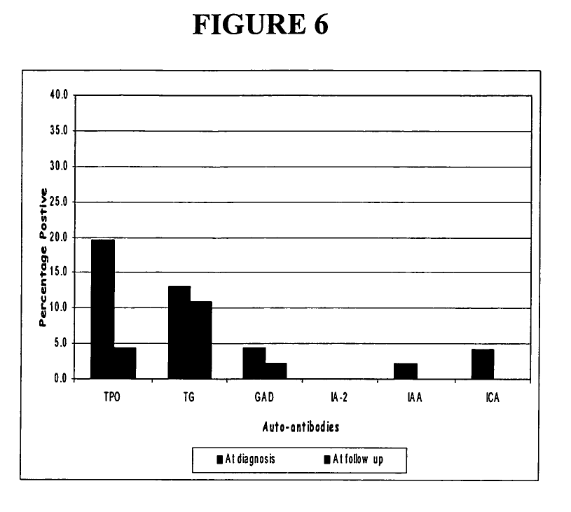

FIG. 7 is a line graph showing the results of an assay for auto-antibodies against thyreoperoxidase (TPO) in subjects that responded to the GFD.

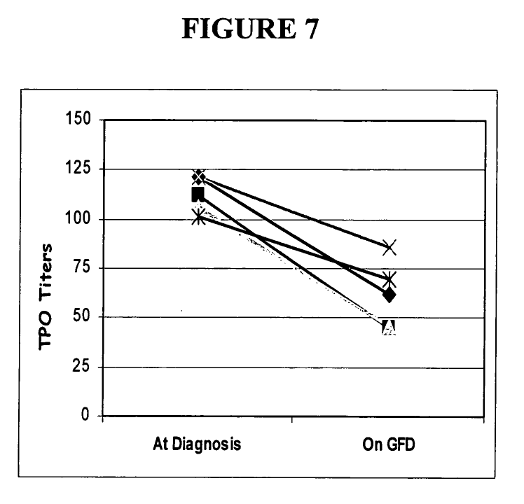

FIG. 8 is a line graph showing the results of an assay for zonulin in subjects that responded to the GFD and initially had TPO auto-antibodies.

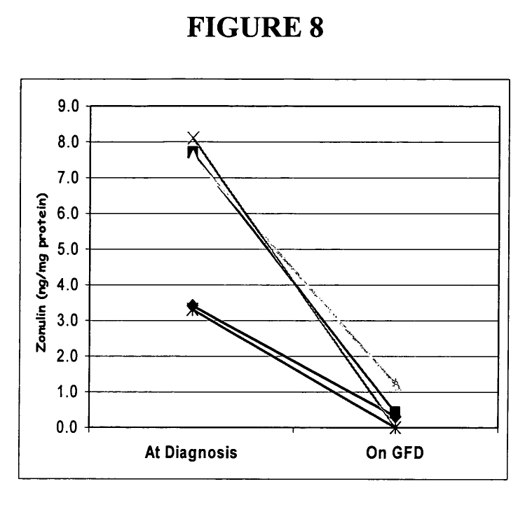

FIG. 9 is a line graph showing the results of an assay for auto-antibodies against thyreoperoxidase (TG) in subjects that responded to the GFD.

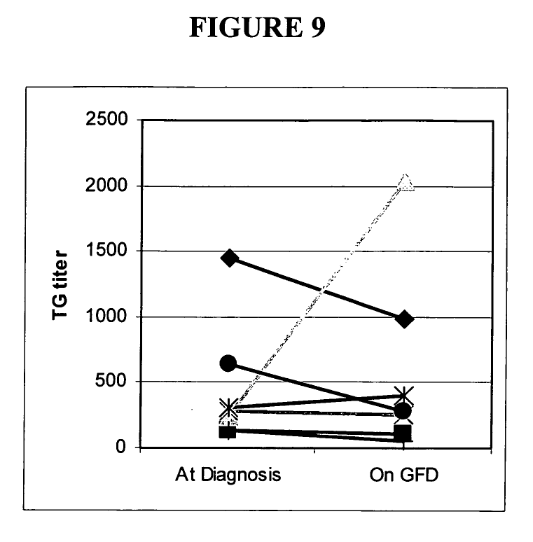

FIG. 10 is a line graph showing the results of an assay for serum zonulin levels in subjects that responded to the GFD and initially had TG auto-antibodies.

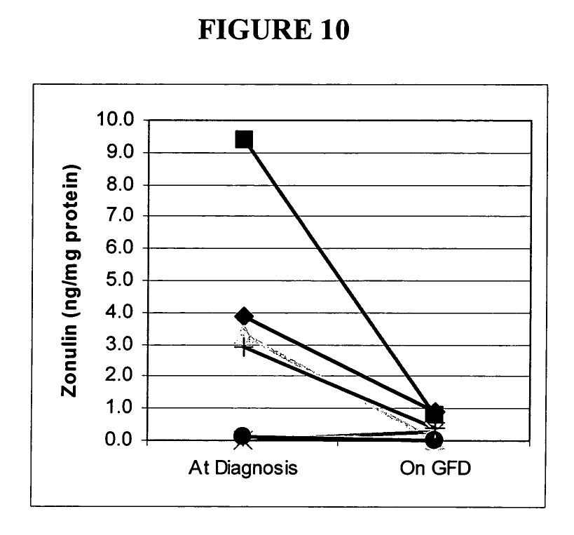

FIG. 11 is a line graph showing the results of an assay for auto-antibodies against islet cells (ICA) in subjects that responded to the GFD.

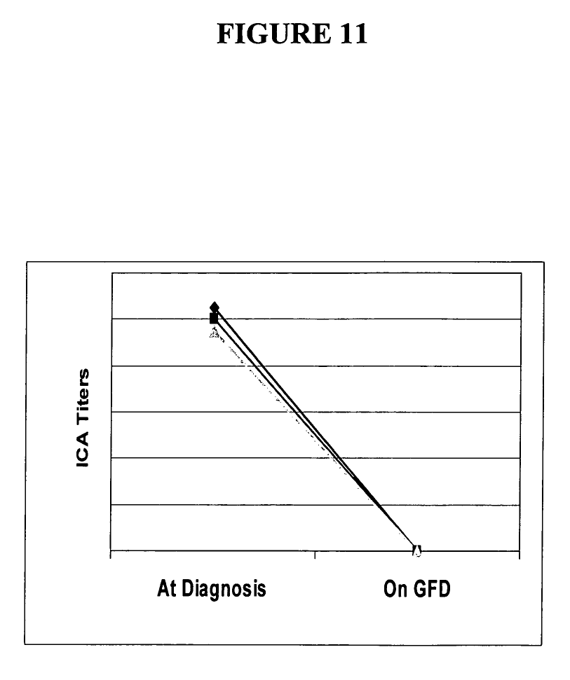

FIG. 12 is a line graph showing the results of an assay for serum zonulin levels in subjects that responded to the GFD and initially had ICA auto-antibodies.

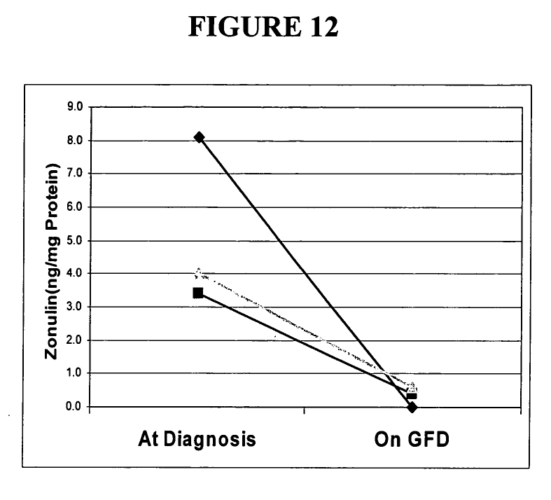

FIG. 13 is a line graph showing the results of an assay for auto-antibodies against glutamic acid decarboxylase (GAD) in subjects that responded to the GFD.

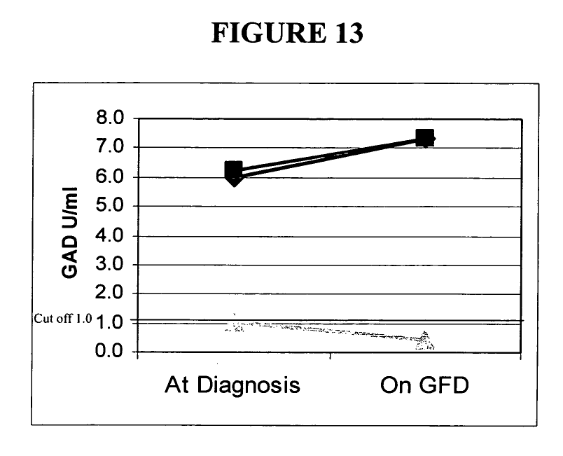

FIG. 14 is a line graph showing the results of an assay for serum zonulin levels in subjects that responded to the GFD and initially had GAD auto-antibodies.

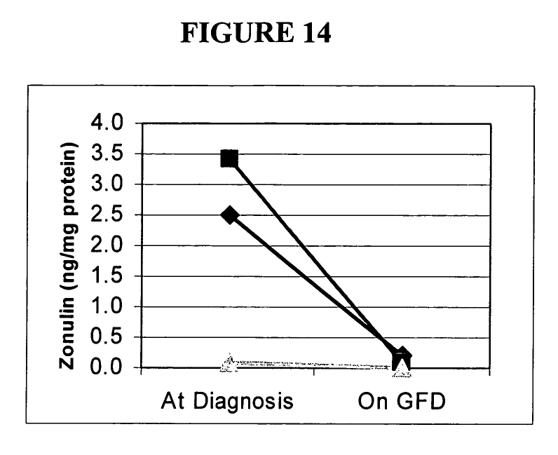

FIG. 15 is a pie chart showing percent of subjects who tested positive for auto-antibodies in relation to HLA type.

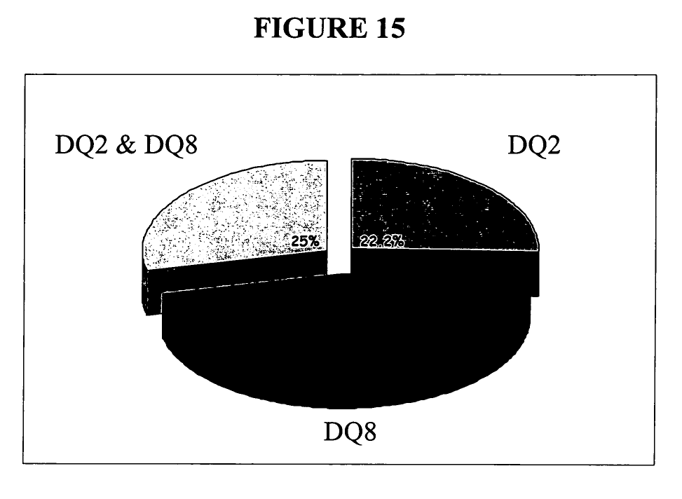

FIG. 16 is a bar graph showing prevalence of auto-antibodies in relation to age of subjects.

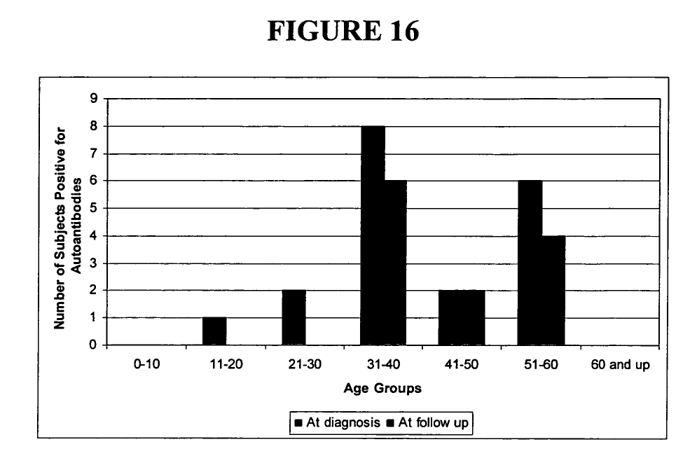

FIG. 17 shows the results of serum zonulin level assays in serum obtained from patients with the indicated autoimmune diseases HC (healthy control), CD (celiac disease), AIH (autoimmune hepatitis), PBC (primary biliary cirrhosis), DM (degenerative myelopathy), APS-1 (autoimmune polyglandular syndrome type 1), MS (multiple sclerosis).

### DETAILED DESCRIPTION OF THE INVENTION

As used herein a subject is any animal, e.g., mammal, that receives a treatment. Subjects include, but are not limited to, humans.

Those of skill in the art will appreciate the present invention may be used to evaluate the effectiveness of treatment of any disease characterized by an elevated serum zonulin level. In particular, the present invention may be used to assess the effectiveness of treatment of autoimmune diseases associated with an increase in serum zonulin levels, for example, celiac disease, primary biliary cirrhosis, and degenerative myelopathy.

Materials and methods of the invention may be used to evaluate the effectiveness of a treatment of celiac disease. For example, a serum zonulin level may be determined for a subject in need of a treatment for celiac disease prior to or concomitant with the beginning of a treatment regimen. At appropriate times during and/or after the course of treatment, one or more additional serum zonulin levels may be determined and compared to the serum zonulin level obtained at the beginning of treatment. Thus, in some embodiments of the invention, methods of the invention may entail repeated acquisition of samples from subjects receiving or who have received treatments and determining zonulin levels in the samples. Methods of the invention may also entail comparing the zonulin levels in the samples to that of a reference sample which may be from the same subject (for example, from a sample taken at the start of the treatment) or from a different source. A decrease in serum zonulin levels is indicative of an amelioration of the condition and indicates that the treatment is effective.

Materials and methods of the invention may be used to evaluate the effectiveness of a treatment of a disease associated with celiac disease. Those of skill in the art are aware that numerous diseases are associated with celiac disease (see, for example, Table 1 in Lee and Green, Current Opinion in Rheumatology 2006, 18:101-107 at page 104) and materials and methods of the invention may be used to evaluate the effectiveness of treatment of celiac associated diseases. Diseases associated with celiac disease may include: neurologic diseases such as peripheral neuropathy, cerebellar ataxia, epilepsy, and migraines; endocrine diseases such as Type 1 diabetes mellitus, autoimmune thyroid disorders, Addison's disease, and alopecia areata; cardiac diseases such as idiopathic dilated cardiomyopathy and autoimmune myocarditis; hepatic diseases such as primary biliary cirrhosis, autoimmune hepatitis, and autoimmune cholangitis; rheumatologic diseases such as oligoarticular arthritis, juvenile arthritis, systemic lupus erythmatosus, and Sjogren's syndrome; and other diseases such as anemia, osteoporosis or osteopenia, Turner syndrome, Down syndrome, dental enamel defects, sarcoidosis, lactose intolerance, dermatitis herpetiformis (a burning, itching, blistering rash), and other skin disorders, unexplained infertility, miscarriage, and recurrent acute pancreatitis.

Materials and methods of the invention may be used to evaluate the effectiveness of treatment for a variety of conditions including, but not limited to, autoimmune diseases. Examples of autoimmune diseases include, but are not limited to, IgA nephropathy, Wegener's granulomatosis, multiple sclerosis, scleroderma, systemic sclerosis, rheumatoid arthritis, Crohn's disease, lupus erythematosus, Hashimoto's thyroiditis (underactive thyroid), Graves' disease (overactive thyroid), autoimmune inner ear disease, bullous pemphigoid, Devic's syndrome, Goodpasture's syndrome, Lambert-Eaton myasthenic syndrome (LEMS), autoimmune lymphproliferative syndrome (ALPS), paraneoplastic syndromes, and polyglandular autoimmune syndromes (PGA).

Materials and methods of the invention may be used to evaluate the effectiveness of a treatment of gastrointestinal inflammation that gives rise to increased intestinal permeability. Thus, the present invention is useful, e.g., in the evaluation of treatment of intestinal conditions that cause protein losing enteropathy. Protein losing enteropathy may arise due to:

infection, e.g., Clostridium difficile infection, enterocolitis, shigellosis, viral gastroenteritis, parasite infestation, bacterial overgrowth, Whipple's disease;

diseases with mucosal erosion or ulcerations, e.g., gastritis, gastric cancer, collagenous colitis, inflammatory bowel disease; and

mucosal diseases without ulceration, e.g., Menetrier's disease, celiac disease, eosinophilic gastroenteritis.

The present invention also includes kits for evaluating the effectiveness of a treatment as well as kits for determining zonulin concentration, for example, from serum. Kits of the invention will typically comprise one or more containers containing one or more reagents useful in the practice of the invention. Reagents useful in the practice of the invention include, but are not limited to, buffers, buffer salts, metal ions, chromogenic compounds, antibodies, enzymes, fluorescent compounds and the like. Kits of the invention may comprise one or more containers containing zonulin, ΔG, or other compound that may be used as a reference standard. Kits of the invention may comprise containers containing one or more antibodies wherein the antibodies are conjugated to a detectable moiety. Detectable moieties may be any known to those skilled in the art, for example, enzymes (e.g., peroxidase, luciferase), other proteins (e.g., green fluorescent protein), optically detectable compounds (e.g., fluorophores, chromophores), members of a binding pair (e.g., biotin/streptavidin, digoxigenin/anit-digoxigenin), or any other detectable moiety known to those skilled in the art. When a kit of the invention comprises an enzyme, such a kit may comprise one or more containers containing substrate for the enzyme.

The following examples are provided for illustrative purposes only, and are in no way intended to limit the scope of the present invention.

### EXAMPLE 1

Patients and Methods:

They were 54 patients diagnosed with CD (20 M and 34 F; mean age: 39y; biopsy-proven: 42/54). Associated AD were found in 8 subjects (2 Type 1 diabetes, 1 Graves's disease, 5 rheumatoid arthritis).

Serum samples were collected at diagnoses and after a mean period of 17 months of GFD (range 10-49). All serum samples were measured for auto-antibodies related to CD (anti-transglutaminase—tTG, anti-endomysial—EMA), Type 1 diabetes (IA-2: tyrosine phosphates, IAA: anti-insulin antibodies, GAD: glutamic acid decarboxylase), thyroiditis (TPO: thyreoperoxidase antibodies, TG: thyreoglobulin antibodies), and zonulin levels.

Serum zonulin measurement by sandwich enzyme-linked immunosorbent assay. Zonulin sandwich enzyme-linked immunosorbent assay (ELISA) was performed as described in E1 Azmar et al. Gastroenterology 123:1607-1615, 2002 with minor modifications.

Briefly, plastic microtiter plates (Costar, Cambridge, Mass.) were coated with rabbit zonulin cross-reacting anti-Zonula occludens toxin (Zot) derivative ΔG IgG antibodies (10 μg/ml in 0.1 mol/l sodium carbonate buffer, pH 9.0). These antibodies were prepared by immunizing a rabbit with ΔG using standard protocols.

After overnight incubation at 4° C., plates were washed four times in Tris buffered saline 0.05% Tween 20 (TBS-T) and blocked by incubation for 1 h at 37° C. with TBS-T. After four TBS-T washes, five ΔG serial standards (50, 25, 12.5, 6.2, 3.1, and 0 ng/ml) and patient sera samples (1:101 dilution in TBS-T) were added and incubated overnight at 4° C. After four washes with Tris buffered saline 0.2% Tween 20 buffer, plates were incubated with biotinylated anti-Zot IgG antibodies (U.S. Pat. No. 5,945,510) for 4 h at 4° C. and contacted with streptavidin-conjugated alkaline phosphatase. A color reaction was developed by using a commercial kit (ELISA amplification kit; Invitrogen). The absorbance at 495 nm was measured with a microplate auto-reader (Molecular Devices Thermomax Microplate Reader).

Auto antibodies were detected using commercially available kits: anti-(human)-transglutaminase (anti-tTG) enzyme-linked immunosorbent assay (ELISA), Scimedx Corporation, NJ; anti-endomysial (anti-EMA) indirect immunofluorescence antibody assay (IFA), using tissue from monkey esophagus, Scimedx Corporation, NJ; anti-tyrosine phosphates (anti IA-2) radioimmunoassay (RIA), Kronus Boise Research Center, Idaho, anti-thyroid peroxidase antibodies (anti-TPO) enzyme immunoassay (EIA), Scimedx Corporation, NJ; anti-thyroglobulin (anti-TG) EIA, Scimedx Corporation, NJ; anti-islet cell antibodies (anti-ICA) IFA, using tissue from monkey pancreas, Scimedx Corporation, NJ, anti-insulin antibodies (IAA) RIA, Kronus Boise Research Center, Idaho; and anti-glutamic acid decarboxylase (anti-GAD) RIA, Kronus Boise Research Center, Idaho.

Patients having a CD diagnosis showed increased serum zonulin in 76% and auto-antibodies were detected in 39% (TPO: 21.7%, TG 19.6%, GAD 6.5%, ICA 4.4% and IA-2 2.5%). After GFD, EMA and zonulin remained altered in 13% of patients, and tTG in 35% of the subjects. Some auto-antibodies decreased (TPO: 10.9%, GAD 4.4%), while other remained unchanged (TG 23.9%, ICA 4.4%, and IA-2 2.2%). Seven out of 53 patients did not start the GFD. These subjects had altered zonulin, EMA, and tTG and 14% of them were auto-antibodies positive. In these subjects, both zonulin levels and serum auto-antibodies did not change at the follow-up evaluation. Conclusions: Untreated CD typically show zonulin up-regulation and increased prevalence of serum auto-antibodies. After treatment with GFD, serum zonulin levels tend to normalize, a situation that is associated with a decreased prevalence of some auto-antibodies (especially TPO). These results indirectly suggest that recovery of the intestinal barrier function can decrease the risk of associated autoimmune phenomena. These results also suggest that if the GFD is implemented early (less 30 years of age) the auto-antibodies will seroconvert, suggesting a possible protective roll against autoimmunity co-morbidity, if CD is diagnosed early and started on a GFD.

### EXMAPLE 2

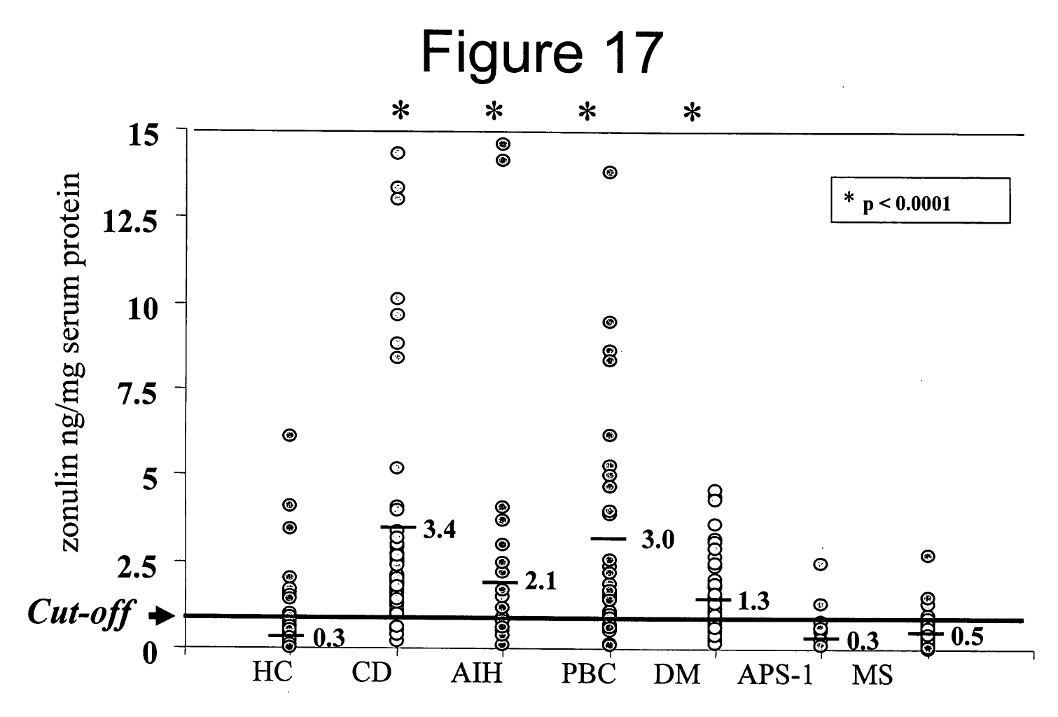

FIG. 17 shows the results of serum zonulin level assays in serum obtained from patients with the indicated autoimmune diseases HC (healthy control), CD (celiac disease), AIH (autoimmune hepatitis), PBC (primary biliary cirrhosis), DM (degenerative myelopathy), APS-1 (autoimmune polyglandular syndrome type 1), MS (multiple sclerosis). Celiac disease, autoimmune hepatitis, primary biliary cirrhosis and degenerative myelopathy are all associated with elevated serum zonulin levels.

While the invention has been described in detail, and with reference to specific embodiments thereof, it will be apparent to one of ordinary skill in the art that various changes and modifications can be made therein without departing from the spirit and scope thereof and such changes and modifications may be practiced within the scope of the appended claims. All patents and publications herein are incorporated by reference to the same extent as if each individual publication was specifically and individually indicated to be incorporated by reference in their entirety.

What is claimed is:

1. A method of assessing the effectiveness of a treatment of a condition in a subject in need of such treatment, comprising:
obtaining a sample from the subject; and
determining zonulin concentration in the sample.

2. A method according to claim 1, wherein the sample is a blood sample.

3. A method according to claim 2, further comprising:
isolating serum from the sample; and
determining zonulin concentration in the serum.

4. A method according to claim 1, wherein the condition is associated with an increase in epithelial permeability.

5. A method according to claim 4, wherein the increase in epithelial activity is associated with an increase in tight junction permeability.

6. A method according to claim 1, wherein the condition is an autoimmune disease associated with an elevated serum zonulin level.

7. A method according to claim 1, wherein the condition is celiac disease.

8. A method according to claim 1, wherein the condition is a disease associated with celiac disease.

9. A method according to claim 1, wherein the disease associated with celiac disease is selected from a group consisitng of peripheral neuropathy, cerebellar ataxia, epilepsy, migraines, Type 1 diabetes mellitus, autoimmune thyroid disorders, Addison's disease, alopecia areata, idiopathic dilated cardiomyopathy, autoimmune myocarditis, primary biliary cirrhosis, autoimmune hepatitis, autoimmune cholangitis, oligoarticular arthritis, juvenile arthritis, systemic lupus erythmatosus, Sjogren's syndrome, anemia, osteoporosis, osteopenia, Turner syndrome, Down syndrome, dental enamel defects, sarcoidosis, lactose intolerance, dermatitis herpetiformis, unexplained infertility, miscarriage, and recurrent acute pancreatitis.

10. A method according to claim 1, wherein determining zonulin concentration comprises:
contacting the sample with a first antibody that binds to zonulin under binding conditions;
contacting the bound sample with a second antibody that binds zonulin under binding conditions; and
detecting the presence of bound second antibody.

11. A method according to claim 10, wherein at least one antibody was raised against a protein comprising a fragment of zonula occludens toxin.

12. A method according to claim 10, wherein the first antibody was raised against a protein comprising a fragment of zonula occludens toxin.

13. A method according to claim 10, wherein the first antibody was raised against a protein comprising ΔG fragment of zonula occludens toxin.

14. A method according to claim 13, wherein the ΔG fragment comprises an N-terminal six histidine tag.

15. A method according to claim 10, wherein at least one antibody was raised against a protein comprising zonula occludens toxin.

16. A method according to claim 10, wherein the second antibody was raised against a protein comprising zonula occludens toxin.

17. A method according to claim 16, wherein the second antibody comprises a detectable moiety.

18. A method according to claim 17, wherein the detectable moiety is biotin.

19. A method for assessing the efficacy of a treatment for celiac disease, comprising:
obtaining a sample from the subject; and
determining zonulin concentration in the sample.

20. A method according to claim 19, wherein the sample is a blood sample.

21. A method according to claim 19, further comprising:
isolating serum from the sample; and
determining zonulin concentration in the serum.

22. A method according to claim 21, wherein determining zonulin concentration comprises:
contacting the sample with a first antibody that binds to zonulin under binding conditions;
contacting the bound sample with a second antibody that binds zonulin under binding conditions; and
detecting the presence of bound second antibody.

23. A method according to claim 22, wherein at least one antibody was raised against a protein comprising a fragment of zonula occludens toxin.

24. A method according to claim 23, wherein the first antibody was raised against a protein comprising a fragment of zonula occludens toxin.

25. A method according to claim 22, wherein the first antibody was raised against a protein comprising ΔG fragment of zonula occludens toxin.

26. A method according to claim 25, wherein the ΔG fragment comprises an N-terminal six histidine tag.

27. A method according to claim 22, wherein at least one antibody was raised against a protein comprising zonula occludens toxin.

28. A method according to claim 22, wherein the second antibody was raised against a protein comprising zonula occludens toxin.

29. A method according to claim 28, wherein the second antibody comprises a detectable moiety.

30. A method according to claim 29, wherein the detectable moiety is biotin.

31. A kit for the evaluation of the efficacy of a treatment comprising:
means for detecting zonulin.

32. A kit according to claim 31, wherein the means for detecting zonulin comprises:
a container containing a first antibody and a container containing a second antibody.

33. A kit according to claim 31, further comprising a container containing ΔG fragment of zonula occludens toxin.

34. A kit according to claim 31, wherein at least one antibody was raised against a protein comprising a fragment of zonula occludens toxin.

35. A kit according to claim 31, wherein the first antibody was raised against a protein comprising a fragment of zonula occludens toxin.

36. A kit according to claim 31, wherein the first antibody was raised against a protein comprising ΔG fragment of zonula occludens toxin.

37. A kit according to claim 36, wherein the ΔG fragment comprises an N-terminal six histidine tag.

38. A kit according to claim 31, wherein at least one antibody was raised against a protein comprising zonula occludens toxin.

39. A kit according to claim 31, wherein the second antibody was raised against a protein comprising zonula occludens toxin.

40. A kit according to claim 39, wherein the second antibody comprises a detectable moiety.

41. A kit according to claim 40, wherein the detectable moiety is biotin.

42. A kit according to claim 31, further comprising a container containing zonulin.

43. A kit for the evaluation of the efficacy of a treatment for celiac disease, comprising:
means for detecting zonulin.

44. A kit according to claim 43, wherein the means for detecting zonulin comprises:
a container containing a first antibody and a container containing a second antibody.

45. A kit according to claim 43, further comprising a container containing ΔG fragment of zonula occludens toxin.

46. A kit according to claim 43, wherein at least one antibody was raised against a protein comprising a fragment of zonula occludens toxin.

47. A kit according to claim 43, wherein the first antibody was raised against a protein comprising a fragment of zonula occludens toxin.

48. A kit according to claim 43, wherein the first antibody was raised against a protein comprising ΔG fragment of zonula occludens toxin.

49. A kit according to claim 48, wherein the ΔG fragment comprises an N-terminal six histidine tag.

50. A kit according to claim 43, wherein at least one antibody was raised against a protein comprising zonula occludens toxin.

51. A kit according to claim 43, wherein the second antibody was raised against a protein comprising zonula occludens toxin.

52. A kit according to claim 51, wherein the second antibody comprises a detectable moiety.

53. A kit according to claim 52, wherein the detectable moiety is biotin.

54. A kit according to claim 43, further comprising a container containing zonulin.
